(19) United States
(12) Patent Application Publication
(10) Pub. No.: US 2010/0159538 A1
(43) Pub. Date: Jun. 24, 2010

(54) METHOD FOR THE CHEMOENZYMATIC PRODUCTION OF FATTY ACID ESTERS

(75) Inventors: Sabine Both, Neuss (DE); Ulrich Schorken, Deutschland (DE); Carolin Meyer, Deutschland (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/063,547

(22) PCT Filed: Aug. 2, 2006
(86) PCT No.: PCT/EP2006/007633

§ 371 Date: Feb. 11, 2008

(30) Foreign Application Priority Data
Aug. 11, 2005 (DE) 10 2005 037 989.3

(51) Int. Cl. C12P 7/64

(52) U.S. Cl. 435/134

(57) ABSTRACT

A chemoenzymatic process for the production of fatty acid esters comprising partially esterifying one or more fatty acid esters, under mild temperatures with an enzymatic catalyst, and optionally in the presence of one or more inert solvents, with a 1- to 5-fold molar excess of one or more water-containing aliphatic alcohols with boiling points between 60° C. and 120° C., then removing the water and unreacted alcohol(s) from the resulting pre-esterification product, followed by additional esterification up to 99.7%, chemically-catalyzed with, e.g., an acid or tin salt at slightly higher temperatures, optionally using one or more inert solvents, with a 1- to 4-fold molar excess of the same one or more aliphatic alcohols as employed in the preliminary esterification step.

### RELATED APPLICATIONS

This application is filed under 35 U.S.C. §371, claiming priority from PCT/EP2006/007633 filed Aug. 2, 2006, which claims priority from DE 10 2005 037 989.3 filed Aug. 11, 2005; the entire contents of each application are incorporated herein by reference.

### FIELD OF THE INVENTION

This invention relates to a process for the chemo-enzymatically catalyzed production of fatty acid esters whose alcohol components are aliphatic alcohols having a boiling point between 60 and 120° C.

### BACKGROUND AND RELATED ART

Enzymes are being increasingly used as catalysts in chemical and biochemical synthesis. In many cases, esterases and especially lipases (EC 3.1.1.3) are already being used in industrial fat-splitting, esterification and transesterification processes by virtue of the often milder reaction conditions employed with enzymes. To isolate the enzymes, fermentation of each of the different microorganisms which produce them is followed by an expensive purification process. The effectiveness of these catalysts is often offset by the high costs of production and isolation, so that research groups are constantly striving to increase the yields or the productivity of the enzymes.

Suitable biocatalytic synthesis processes are described, for example, in K. Drauz and H. Waldmann, Enzyme Catalysis in Organic Synthesis, WILEY-VCH, Vol. I to III, 2002; and U. T. Bornscheuer, R. J. Kazlauskas in Hydrolases in Organic Synthesis. The industrial transformation of biocatalytic processes is described by A. Liese, K. Seelbach and C. Wandrey in Industrial Biotransformations, WILEY-VCH, 2002.

Enzyme-catalyzed esterifications are known, as is the use of immobilized enzymes t to improve cost efficiency in a process and microencapsulation, for example, of enzymes or microorganisms to stabilize them and permit their use several times. However, the disadvantage of biocatalytic reactions often still lies in the availability and stability of the catalysts involved in the process.

Chemically catalyzed esterifications, such as acidic catalysis for example, are also known. In such esterification processes, a stoichiometric amount of water is released and has to be removed in order to steer the reaction towards the ester product.

The disadvantage of the purely chemically or purely enzymatically catalyzed esterification of fatty acids with aliphatic alcohols having a boiling point of 60 to 120° C. lies in the large volume of distillate which contains varying amounts of water according to the conversion level. Even in the enzyme-catalyzed processes, the water released at high conversion levels has to be removed from the reaction mixture, generally in vacuo and at relatively high temperatures, which can result in deactivation of the enzyme.

Alcohols which have a boiling point below that of water or which form a low-boiling azeotrope with water (such as ethanol for example) are particularly difficult to work up. Complicated processes, such as membrane separation, molecular sieve drying or azeotropic distillation using entraining agents, have to be used for this purposes, resulting in high process costs.

In addition, if the purely chemically catalyzed process is used, long reactor possession times are the outcome. The high temperatures involved adversely affect the color value of the product to a considerable extent by comparison with enzyme-catalyzed esterifications.

Accordingly, the problem addressed by the present invention was to provide a process in which fatty acids could be esterified with aliphatic alcohols having a boiling point of 60 to 120° C. without any need for expensive drying of the alcohol component. In addition, the process would be cost-efficient and recyclable. The enzymes would only be minimally stability-impaired.

### BRIEF SUMMARY OF THE INVENTION

The present invention relates to a process for the production of one or more fatty acid esters wherein:
(a) one or more fatty acids are treated in the presence of one or more esterases with one or more water-containing aliphatic alcohols, each alcohol having a boiling point between 60 and 120° C., to produce a pre-esterification product;
(b) water and unreacted alcohol(s) are removed from the pre-esterification product,
(c) the pre-esterification product is subjected to a second, chemically catalyzed esterification with the same aliphatic alcohol(s) as in step (a) and one or more chemical catalysts; and
(d) the water-containing aliphatic alcohol(s) removed from step (c) is/are re-used in step (a).

### DETAILED DESCRIPTION OF THE INVENTION

The instant invention is directed to a process for producing one or more fatty acid esters, wherein:
(a) one or more fatty acids are treated in the presence of one or more esterases with one or more water-containing aliphatic alcohols, each alcohol having a boiling point between 60 and 120° C., to produce a pre-esterification product;
(b) water and unreacted alcohol(s) are removed from the pre-esterification product;
(c) the pre-esterification product is subjected to a second, chemically-catalyzed esterification with the same aliphatic alcohol(s) as in step (a) and one or more chemical catalysts; and
(d) the water-containing aliphatic alcohol(s) removed from step (c) is/are re-used in step (a).

In another embodiment, water is removed by phase separation during the pre-esterification step (a). In a preferred embodiment of the process according to the invention, an inert organic solvent is added to the enzymatic reaction.

It has surprisingly been found that the process according to the invention eliminates the need to work up the water-containing component of aliphatic alcohol(s) with a boiling point between 60 and 120° C. which forms a low-boiling azeotrope with water. The water-containing alcohol released in the chemical esterification can readily be used in the enzymatic pre-esterification because the enzymes catalyze an esterification at low temperatures reaching into the reaction equilibrium which lies far on the ester side. Since the reaction is carried out at low temperatures, no distillate accumulates. On completion of the preliminary reaction, the water and remaining alcohol(s) are removed and discarded. Since the reaction proceeds strongly in the ester synthesis direction, the loss of low-boiling alcohol(s) is minimal. The reaction can be further driven in the ester direction by the addition of inert solvents such as, for example, hexane, iso-octane or n-octane, still further reducing the loss of low-boiling alcohol(s). This reaction eliminates the need for complicated, energy-intensive working up of the water-containing alcohol(s), representing a saving that provides an economic and ecological advantage.

The advantage of the process according to the invention is that it is a chemo-enzymatic process. In a first stage, the fatty acid is esterified with an aliphatic alcohol/water mixture to a partial conversion. On completion of the reaction, most of the water of reaction can be removed from the product mixture by phase separation. This step is carried out at mild temperatures with defined water/alcohol/ester compositions. The removal of water is improved by addition of a solvent, for example n-octane, in the enzymatic stage. On completion of the enzymatic stage, the solvent, unreacted alcohol(s) and water which has not been removed are distilled off. The partly reacted material remaining is then delivered to a second esterification stage which is catalyzed, for example, by an acid or a tin salt and continued to a conversion of 99 to 99.7%. The alcohol(s)/water of reaction distillate is collected and completely recycled to the first, enzyme-catalyzed pre-esterification stage. Through the combination of both processes and the removal of water by separation in the enzyme-catalyzed stage, the process is highly synergistic. In addition, it is possible by employing the reaction according to the invention, with only slight variation of the conditions, to produce a very broad range of various products in better yields and under more moderate conditions than is possible by the processes known from the prior art.

Some of the conditions for the enzymatic pre-esterification in step (a) include:
use of an alcohol/water mixture with a water content of 0.1 to 50%, the pure alcohol having a boiling point between 60 and 120° C.;
optional use of an inert solvent, such as n-octane for example, for improved water removal in step (b) and for reducing the enzymatic catalysis temperature;
1 to 5-fold molar excess of aliphatic alcohol(s) each of which has a boiling point between 60 and 120° C., a 1.1-fold excess being preferred;
temperature between 20 and 70° C.; and
preferably normal pressure.

All other conditions and particularly the preferred conditions are described elsewhere herein.

Final conversions of 50 to 85% are reached in the pre-esterification, depending on the reaction time. For a conversion of 80%, the reaction time is 8 to 16 hours, depending on the carrier material, the starting quantity of water and the fatty acid used.

Some of the conditions for the post-esterification under chemically catalyzed conditions include:
use of one or more aliphatic alcohol(s) with an alcohol content of at least 95%;
use of the fatty acid/fatty acid ester/alcohol(s) mixture from the pre-esterification, the solvent optionally added and the azeotrope of water and aliphatic alcohol(s) being distilled off beforehand;
1- to 4-fold molar excess of aliphatic alcohol(s), each of which has a boiling point between 60 and 120° C., a 1-fold excess being preferred;
temperature between 150 and 250° C.;
the pressure is intended to be adjusted via a pressure gradient from 5 bar at the beginning of the reaction to 1 bar later. Vacuum is applied towards the end of the reaction in order to separate the product mixture from the unreacted aliphatic alcohol(s);
suitable catalysts preferably include any esterification catalysts, e.g., tin(II) compounds, zinc compounds, sulfuric acid, p-toluenesulfonic acid or acidic ion exchangers; and
the reaction time for the chemically catalyzed reaction is only 10 to 12 hours and is thus reduced by at least 50% compared with the purely chemically catalyzed reaction without enzymatic pre-esterification.

The fatty acids used in the process according to the invention are carboxylic acids with the general formula R—COOH where R is a linear or branched, alkyl or alkenyl group, that are optionally hydroxysubstituted and that have 6 to 32 carbon atoms, and contain up to six conjugated or unconjugated double bonds.

In one particular embodiment of the process according to the invention, the one or more fatty acids used is/are selected from di- and/or polycarboxylic acids with linear or branched, alkyl or alkenyl chains, optionally hydroxysubstituted and containing 2 to 32 carbon atoms.

The fatty acids used in the process according to the invention are selected from the group consisting of caproic acid, oenanthic acid, caprylic acid, pelargonic acid, capric acid, lauric acid, lauroleic acid, myristic acid, myristoleic acid, palmitic acid, palmitoleic acid, stearic acid, petroselic acid, petroselaidic acid, oleic acid, elaidic acid, ricinoleic acid, linoleic acid, linolaidic acid, linolenic acid, elaeostearic acid, arachic acid, gadoleic acid, arachidonic acid, behenic acid, erucic acid, brassidic acid, clupanodonic acid, lignoceric acid, cerotic acid, melissic acid, eicosapentaenoic acid, docosahexaenoic acid, conjugated linoleic acid, isostearic acids, 2-ethylhexanoic acid and mixtures of two or more thereof. Myristic acid, oleic acid, lauric acid and/or palmitic acid are particularly preferred for the process according to the invention.

In a preferred embodiment, the molar ratio between the fatty acid(s) and the aliphatic alcohol(s) used in the process according to the invention differs only very slightly from 1 and, more particularly, is in the range from 0.8 to 1.2, as this results in the highest yields of required product.

Basically, suitable aliphatic alcohols which are added during the pre- and post-esterifications are any fatty alcohols which have a boiling point between 60 and 120° C., i.e. for example alcohols containing 1 to 4 carbon atoms, such as methanol (boiling point 64° C.), ethanol (boiling point 78° C.), propanol (boiling point 97° C.), isopropyl alcohol (boiling point 82° C.) and 1-butanol (boiling point 118° C.), isobutyl alcohol (boiling point 108° C.), sec.butyl alcohol (boiling point 99° C.), tert.butyl alcohol (boiling point 83° C.), and mixtures of two or more thereof. Alcohols with a boiling point between 60 and 100° C. are particularly preferred for the process according to the invention, with isopropyl alcohol being more particularly preferred.

Among the enzymes used in accordance with the invention, enzymes from the group of esterases and especially the lipases are preferred and may be used either individually or in combination of several enzymes.

Esterases from organisms selected from the group consisting of Thermomyces lanugenosus, Candida antarctica A, Candida antarctica B, Rhizomucor miehei, Candida cylindracea, Rhizopus javanicus, Porcine pancreas, Aspergillus niger, Candida rugosa, Mucor javanicus, Pseudomonas fluorescens, Rhizopus oryzae, Pseudomonas sp., Chromobacterium viscosum, Fusarium oxysporum and Penicilium camenberti, and lipases from the organisms mentioned, preferably the lipase from Candida antarctica B, are particularly preferred enzymes for the biocatalysts.

The enzymes to be used in accordance with the invention may be used in different forms, with any of the presentation forms of enzymes familiar to the expert being used. The enzymes are preferably used in pure form or as a technical enzyme preparation either immobilized on a carrier material and/or in solution, more particularly in aqueous solution, and are re-used in repeated batches. Immobilized enzymes adsorbed onto hydrophobic carriers, such as for example polystyrene, polyacrylamide or polypropylene carriers, are particularly preferred.

In a particularly preferred embodiment, the esterase and especially the lipase are used in a stabilized form obtained by chemical modification with crosslinking reagents, more particularly glutaraldehyde, or by chemical surface modification, for example with octanal.

The reaction conditions according to the invention for the biocatalytic reaction are dependent upon the optimal reaction range of the enzymes selected. More particularly, the conditions are conditions where inter alia the reaction temperature is between 20 and 70° C., preferably between 35 and 55° C. and more particularly between 43 and 45° C.

The following Examples are intended to illustrate the instant invention, without, in any way, limiting it.

### EXAMPLES

### Example 1

### Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.548 mol) myristic acid, 62.5 g (1.04 mol) isopropyl alcohol and 6.25 g deionized water were added to 10 g immobilized enzyme on polypropylene pellets, MP-100 (Candida antarctica B lipase, from Novozymes, adsorbed onto polypropylene carrier, enzyme charge 200 mg technical liquid preparation per g carrier) and stirred at 43° C. After 24 h, a conversion of 55% was obtained. After a conversion of ca. 40%, a relatively heavy water phase containing max. 30% isopropyl alcohol began to separate. It was removed from the product mixture so that the reaction could be re-started. After another 24 h, a final conversion of 70% was obtained, another water phase being formed after a conversion of 57%. Analyses of the composition of the water phase typically showed a maximum isopropyl alcohol content of 10% if no solubilizer was used.

### Example 2

### Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.548 mol) myristic acid, 62.5 g (1.04 mol) isopropyl alcohol and 11 g deionized water were added to 10 g immobilized enzyme on polypropylene pellets, MP-100 (Candida antarctica B lipase, from Novozymes, adsorbed onto polypropylene carrier, enzyme charge 200 mg technical liquid preparation per g carrier) and stirred at 43° C. After 24 h, a conversion of 55% was obtained. After a conversion of ca. 40%, a relatively heavy water phase containing max. 30% isopropyl alcohol began to separate. It was removed from the product mixture so that the reaction could be re-started. After another 24 h, a final conversion of 70% was obtained, another water phase being formed after a conversion of 57%. Analyses of the composition of the water phase typically showed a maximum isopropyl alcohol content of 10% if no solubilizer was used.

### Example 3

### Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.548 mol) myristic acid, 62.5 g (1.04 mol) isopropyl alcohol and 11 g deionized water were added to 10 g immobilized enzyme on polypropylene pellets, MP-100 (Candida antarctica B lipase, from Novozymes, adsorbed onto polypropylene carrier, enzyme charge 200 mg technical liquid preparation per g carrier) and stirred at 53° C. After 24 h, a conversion of 55% was obtained. After a conversion of ca. 40%, a relatively heavy water phase containing max. 30% isopropyl alcohol began to separate. It was removed from the product mixture so that the reaction could be re-started. After another 24 h, a final conversion of 70% was obtained, another water phase being formed after a conversion of 57%. Analyses of the composition of the water phase typically showed a maximum isopropyl alcohol content of 10% if no solubilizer was used.

### Example 4

### Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.548 mol) myristic acid, 62.5 g (1.04 mol) isopropyl alcohol and 3.25 g deionized water were added to 10 g immobilized enzyme on polypropylene powder, MP-100 (Candida antarctica B lipase, from Novozymes, adsorbed onto polypropylene carrier, enzyme charge 500 mg technical liquid preparation per g carrier) and stirred at 60° C. After 8 h, a conversion of 70% was obtained. After a conversion of ca. 60%, a relatively heavy water phase containing max. 30% isopropyl alcohol began to separate. It was removed from the product mixture so that the reaction could be re-started. After another 4 h, a final conversion of 83% was obtained, another water phase being formed. Analyses of the composition of the water phase typically showed a maximum isopropyl alcohol content of 10% if no solubilizer was used.

### Example 5

### Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.548 mol) myristic acid, 62.5 g (1.04 mol) isopropyl alcohol and 11 g deionized water were added to 10 g immobilized enzyme on polypropylene powder, MP-100 (Candida antarctica B lipase, from Novozymes, adsorbed onto polypropylene carrier, enzyme charge 500 mg technical liquid preparation per g carrier) and stirred at 43° C. After 8 h, a conversion of 70% was obtained. After a conversion of ca. 60%, a relatively heavy water phase containing max. 30% isopropyl alcohol began to separate. It was removed from the product mixture so that the reaction could be re-started. After another 4 h, a final conversion of 83% was obtained, another water phase being formed. Analyses of the composition of the water phase typically showed a maximum isopropyl alcohol content of 10% if no solubilizer was used.

### Example 6

### Step 1, Enzymatic Pre-Esterification

7.5 g (32.9 mmol) myristic acid, 2.5 g (41.7 mmol) isopropyl alcohol, 0.2 g deionized water and 10 g octane were added to 2 g immobilized enzyme on polypropylene powder (Candida antarctica B lipase, from Novozymes, adsorbed onto a polypropylene carrier, enzyme charge 500 mg technical liquid preparation per g carrier, crosslinked with glutaraldehyde by standard methods). The mixture was incubated at 45° C. in a stoppered Erlenmeyer flask on a shaker. Samples were taken after 4 h and 24 h and the conversion was determined by measurement of the acid value. After termination of the reaction, the enzyme immobilizate was filtered off and re-used under identical conditions in a new batch. The test was carried out in this form over a period of 35 days.

TABLE 1
activity determination of the enzyme immobilizate

| Reaction | Conversion | Conversion [%] |
|---|---|---|
| (days) | [%] (after 4 h) | (after 24 h) |
| 1 | — | 79.2 |
| 2 | — | 77.1 |
| 3 | — | 76.6 |
| 8 | 80.4 | 81.0 |
| 9 | 82.7 | 84.7 |
| 10 | 82.4 | 81.2 |
| 11 | 81.6 | 82.9 |
| 14 | 82.7 | 80.2 |
| 15 | 81.9 | 82.4 |
| 16 | 80.6 | 82.8 |
| 17 | 81.2 | 82.1 |
| 18 | 81.3 | 85.7 |
| 24 | 77.6 | 79.6 |
| 26 | 80.0 | 81.2 |
| 29 | 81.3 | 82.1 |
| 30 | 78.6 | 81.7 |
| 31 | 79.8 | 82.7 |
| 32 | 80.0 | 84.6 |
| 35 | | 83.6 |

No loss of activity of the enzyme immobilizate was observed over a period of 35 days. Without removal of a reaction product, a conversion of ca. 80% was reached. In each reaction, a water phase clearly demarcated from the organic phase was separated. Analyses of the composition of the water phase typically showed a maximum isopropyl alcohol content of 3-5% if a solubilizer was used.

### Example 7

### Step 1, Enzymatic Pre-Esterification

11.25 g (49.3 mmol) myristic acid, 3.75 g (62.5 mmol) isopropyl alcohol, 0.2 g deionized water and 5 g octane were added to 2 g immobilized enzyme (immobilizate from Example 6 re-used). The mixture was incubated at 45° C. in a stoppered Erlenmeyer flask on a shaker. Samples were taken after 4 h and 24 h and the conversion was determined by measurement of the acid value. After termination of the reaction, the enzyme immobilizate was filtered off and re-used under identical conditions in a new batch. The test was carried out in this form over a period of 115 days.

TABLE 2
activity determination of the enzyme immobilizate - 2nd test

| Reaction | Conversion [%] | Conversion [%] |
|---|---|---|
| (days) | (after 4 h) | (after 24 h) |
| 1 | — | 80.8 |
| 2 | 69.6 | 79.2 |
| 3 | 67.9 | 80.3 |
| 4 | 67.2 | 78.1 |
| 6 | 68.6 | 78.8 |
| 7 | 66.4 | 74.5 |
| 9 | 68.5 | 78.9 |
| 10 | 69.1 | 78.7 |
| 11 | 68.5 | 78.7 |
| 14 | 66.6 | 79.9 |
| 15 | 66.6 | 79.9 |
| 16 | 67.3 | 80.1 |
| 17 | 66.1 | 79.1 |
| 21 | 65.0 | 79.1 |
| 22 | 65.2 | 79.5 |
| 23 | 63.9 | 79.4 |
| 24 | 64.6 | 79.2 |
| 25 | 62.9 | 79.4 |
| 28 | 63.5 | 79.9 |
| 29 | 62.0 | 79.9 |
| 30 | 60.2 | 79.5 |
| 31 | 61.9 | 78.6 |
| 32 | 61.3 | 76.5 |
| 36 | 57.4 | 79.5 |
| 37 | 60.5 | 81.2 |
| 43 | 49.8 | 79.2 |
| 44 | 55.5 | 79.7 |
| 45 | 54.4 | 79.2 |
| 46 | 55.5 | 79.6 |
| 49 | 50.6 | 80.0 |
| 50 | 52.6 | 79.2 |
| 51 | 50.2 | 79.9 |
| 52 | 52.4 | 80.0 |
| 53 | 50.5 | 79.8 |
| 56 | 53.3 | 79.5 |
| 57 | 49.0 | 79.6 |
| 65 | 48.5 | 79.1 |
| 66 | 50.8 | 80.2 |
| 67 | 49.6 | 79.3 |
| 70 | 47.2 | 79.4 |
| 71 | 48.4 | 79.4 |
| 72 | 44.7 | 78.9 |
| 74 | 45.3 | 79.0 |
| 78 | 48.7 | 79.2 |
| 85 | 42.0 | 79.0 |
| 86 | 41.3 | 79.3 |
| 87 | 38.6 | 79.1 |
| 91 | 44.1 | 79.6 |
| 92 | 41.5 | 79.4 |
| 93 | 40.9 | 78.9 |
| 94 | 39.2 | 78.9 |
| 95 | 41.1 | 79.7 |
| 98 | 38.3 | 79.9 |
| 99 | 38.2 | 78.7 |
| 100 | 38.1 | 78.8 |
| 101 | 34.7 | 79.2 |
| 113 | 33.1 | 79.0 |
| 115 | 36.2 | 78.2 |

A loss of activity of the enzyme immobilizate of ca. 50% was observed over a period of 115 days. The half life of the immobilized enzyme under the above conditions was 100-120 days. Without removal of a reaction product, a conversion of ca. 80% was reached. In each reaction, a water phase clearly demarcated from the organic phase was separated. Analyses of the composition of the water phase typically showed a maximum isopropyl alcohol content of 3-5% if a solubilizer was used.

### Example 8

### Step 1, Enzymatic Pre-Esterification

11.25 g (49.3 mmol) myristic acid, 3.75 g (62.5 mmol) isopropyl alcohol, 0.2 g deionized water and 5 g octane were added to 2 g immobilized enzyme on polypropylene powder (from Example 7, re-charged with Candida antarctica lipase, from Novozymes, adsorbed onto polypropylene carrier, enzyme charge 1100 mg technical liquid preparation per g carrier, crosslinked with glutaraldehyde by standard methods). The mixture was incubated at 45° C. in a stoppered Erlenmeyer flask on a shaker. Samples were taken after 4 h and 24 h and the conversion was determined by measurement of the acid value. After termination of the reaction, the enzyme immobilizate was filtered off and re-used under identical conditions in a new batch. The test was carried out in this form over a period of 68 days.

TABLE 3
activity determination of the enzyme immobilizate - 3rd test

| Reaction | Conversion [%] | Conversion [%] |
|---|---|---|
| (days) | (after 4 h) | (after 24 h) |
| 1 | 79.5 | 80.7 |
| 2 | 78.0 | 79.4 |
| 3 | 79.1 | 82.9 |
| 4 | 78.5 | 79.9 |
| 5 | 79.2 | 79.9 |
| 8 | 78.7 | 79.6 |
| 9 | 78.5 | 79.8 |
| 43 | 80.0 | 80.0 |
| 44 | 80.0 | 79.8 |
| 45 | 80.5 | 79.9 |
| 46 | 79.6 | 79.6 |
| 64 | 79.7 | 80.1 |
| 65 | 80.4 | 80.1 |
| 67 | 79.5 | 80.4 |
| 68 | 79.9 | — |

No loss of activity of the enzyme immobilizate was observed over a period of 68 days. Without removal of a reaction product, a conversion of ca. 80% was reached. In each reaction, a water phase clearly demarcated from the organic phase was separated. Analyses of the composition of the water phase typically showed a maximum isopropyl alcohol content of 3-5% if a solubilizer was used.

### Example 9

### Step 1, Enzymatic Pre-Esterification

37.5 g (164.5 mmol) myristic acid, 10.5 g (175.0 mmol) isopropyl alcohol, 2.0 g deionized water and 50 g hexane were added to 5 g immobilized enzyme on polypropylene powder (Candida antarctica B lipase, from Novozymes, adsorbed onto a polypropylene carrier, enzyme charge 500 mg technical liquid preparation per g carrier). The mixture was incubated at 35° C. in a stoppered Erlenmeyer flask on a shaker. Samples were taken after 5 h and 22 h and the conversion was determined by measurement of the acid value. After termination of the reaction, the enzyme immobilizate was filtered off and re-used under identical conditions in a new batch. The test was carried out in this form over a period of 77 days.

TABLE 4
activity determination of the enzyme immobilizate - 4th test

| Reaction | Conversion [%] | Conversion |
|---|---|---|
| (days) | (after 5 h) | [%] (after 22 h) |
| 1 | 61.2 | 72.8 |
| 4 | 51.2 | 80.6 |
| 5 | 42.8 | 82.1 |
| 6 | 40.7 | 78.4 |
| 12 | 47.3 | 83.3 |
| 13 | 39.0 | 82.3 |
| 14 | 43.4 | 82.4 |
| 15 | — | 82.2 |
| 18 | 39.8 | 80.7 |
| 19 | 37.6 | 81.9 |
| 26 | 30.9 | 81.3 |
| 27 | 30.9 | 80.6 |
| 28 | 36.3 | 80.9 |
| 29 | 23.7 | 83.1 |
| 32 | 19.8 | 78.0 |
| 33 | 30.6 | 79.9 |
| 34 | 30.0 | 79.6 |
| 35 | 28.0 | 79.4 |
| 39 | 16.1 | 77.3 |
| 40 | 18.2 | 81.9 |
| 41 | 30.0 | 79.5 |
| 46 | 33.7 | 77.4 |
| 47 | 28.3 | 76.7 |
| 48 | 29.5 | 78.8 |
| 49 | 31.4 | 76.4 |
| 50 | 26.7 | 85.1 |
| 53 | 21.4 | 74.1 |
| 54 | 23.8 | 73.5 |
| 55 | 23.2 | 68.8 |
| 56 | 22.3 | 68.1 |
| 57 | 20.7 | — |
| 62 | 25.0 | 68.8 |
| 63 | 21.3 | 66.2 |
| 64 | 18.7 | — |
| 67 | 15.9 | 60.7 |
| 68 | 10.4 | 54.5 |
| 69 | 16.9 | 57.3 |
| 70 | 14.8 | 56.2 |
| 74 | 15.6 | 55.4 |
| 75 | 13.3 | 52.7 |
| 76 | 10.7 | 51.8 |
| 77 | 11.7 | — |

Deactivation of the enzyme immobilizate corresponding to a half life of the enzyme of ca. 30 days was observed under the above conditions. Without removal of a reaction product, a conversion of ca. 80% was reached. In each reaction, a water phase clearly demarcated from the organic phase was separated. Analyses of the composition of the water phase typically showed a maximum isopropyl alcohol content of 3-5% if a solubilizer was used.

### Example 10

### Esterification with Lauric Acid: Step 1, Enzymatic Pre-Esterification

Test Apparatus: Double-Jacketed Four-Necked Round-Bottomed Flask with Stirrer, Internal Thermometer, Heating Cryostats, and Bottom Outlet Valve

125 g (0.625 mol) lauric acid, 62.5 g (1.04 mol) isopropyl alcohol and 11 g deionized water were added to 10 g immobilized enzyme on polypropylene pellets, MP-100 (Candida antarctica B lipase, from Novozymes, adsorbed onto polypropylene carrier, enzyme charge 200 mg technical liquid preparation per g carrier) and stirred at 43° C. After 24 h, a conversion of 55% was obtained. After a conversion of ca. 40%, a relatively heavy water phase containing max. 30% isopropyl alcohol began to separate. It was removed from the product mixture so that the reaction could be re-started. After another 24 h, a final conversion of 71% was obtained, another water phase being formed after a conversion of 57%.

### Example 11

### Step 1, Enzymatic Pre-Esterification

30 g (150.0 mmol) lauric acid, 9.9 g (165.0 mmol) isopropyl alcohol, 0.5 g deionized water and 8.5 g octane were added to 3 g immobilized enzyme on polypropylene powder (Candida antarctica B lipase, from Novozymes, adsorbed onto a polypropylene carrier, enzyme charge 500 mg techn. liquid preparation per g carrier, crosslinked with glutaraldehyde by standard methods). The mixture was incubated at 45° C. in a stoppered Erlenmeyer flask on a shaker. Samples were taken after 5 h and 24 h and the conversion was determined by measurement of the acid value. After termination of the reaction, the enzyme immobilizate was filtered off and re-used under identical conditions in a new batch. The test was carried out in this form over a period of 9 days.

TABLE 5
activity determination of the enzyme immobilizate - 5th test

| Reaction | Conversion [%] | Conversion [%] |
|---|---|---|
| days | (after 5 h) | (after 24 h) |
| 1 | 46.5 | 75.2 |
| 2 | 49.4 | 74.8 |
| 3 | 45.7 | 74.3 |
| 4 | 45.2 | 74.8 |
| 5 | 41.9 | 75.1 |
| 8 | 43.3 | 74.1 |
| 9 | 42.9 | 74.5 |

A loss of activity of the enzyme immobilizate of ca. 10% was observed over a period of 9 days. Without removal of a reaction product, a conversion of ca. 75% was reached. In each reaction, a water phase clearly demarcated from the organic phase was separated. Analyses of the composition of the water phase typically showed a maximum isopropyl alcohol content of 3-5% if a solubilizer was used.

### Example 12

### Esterification with Palmitic Acid: Step 1, Enzymatic Pre-Esterification

30 g (117.2 mmol) palmitic acid, 7.7 g (128.3 mmol) isopropyl alcohol, 0.5 g deionized water and 8.5 g octane were added to 3 g immobilized enzyme on polypropylene powder (Candida antarctica B lipase, from Novozymes, adsorbed onto a polypropylene carrier, enzyme charge 500 mg techn. liquid preparation per g carrier, crosslinked with glutaraldehyde by standard methods). The mixture was incubated at 45° C. in a stoppered Erlenmeyer flask on a shaker. Samples were taken after 5 h and 24 h and the conversion was determined by measurement of the acid value. After termination of the reaction, the enzyme immobilizate was filtered off and re-used under identical conditions in a new batch. The test was carried out in this form over a period of 9 days.

TABLE 6
activity determination of the enzyme immobiltzate - 6th test

| Reaction | Conversion [%] | Conversion [%] |
|---|---|---|
| days | (after 5 h) | (after 24 h) |
| 1 | 46.8 | 78.4 |
| 2 | 47.8 | 78.3 |
| 3 | 49.4 | 77.8 |
| 4 | 47.1 | 77.9 |
| 5 | 47.8 | 77.9 |
| 8 | 45.2 | 77.4 |
| 9 | 48.1 | 77.8 |

No loss of activity of the enzyme immobilizate was observed over a period of 9 days. Without removal of a reaction product, a conversion of ca. 78% was reached. In each reaction, a water phase clearly demarcated from the organic phase was separated. Analyses of the composition of the water phase typically showed a maximum isopropyl alcohol content of 3-5% if a solubilizer was used.

### Example 13

### Esterification with Oleic Acid: Step 1, Enzymatic Pre-Esterification

110.0 g (390.1 mmol) oleic acid, 26.0 g (433.3 mmol) isopropyl alcohol and 1.0 g deionized water were added to 7 g immobilized enzyme on polypropylene powder (Candida antarctica B lipase, from Novozymes, adsorbed onto a polypropylene carrier, enzyme charge 500 mg technical liquid preparation per g carrier, crosslinked with glutaraldehyde by standard methods). The mixture was incubated at 60° C. in a stoppered Erlenmeyer flask on a shaker. The conversion was determined by measurement of the acid value.

TABLE 7
determination of conversion in the reaction
of oleic acid with isopropyl alcohol

| Time [h] | Conversion [%] |
|---|---|
| 0.4 | 4.7 |
| 1 | 9.4 |
| 2 | 20.1 |
| 4 | 42.8 |
| 6 | 63.0 |
| 8 | 70.1 |
| 24 | 74.4 |

### Example 14

### Stage 2, Chemically Catalyzed Reaction

100 kg pre-esterified mixture (IPM, IPA, octane, myristic acid) were introduced into the reaction vessel. The mixture was heated with stirring to 225° C. in a gentle stream of nitrogen (4 l/h). The distillate accumulating was collected via a dephlegmator (Tₛₑₜ=130° C.) and balanced. After termination of the reaction, the distillate was introduced into the enzymatic pre-esterification. When the temperature was reached, a pressure of 5 bar was initially adjusted. The catalyst, 200 g tin(II) compound, was then introduced. After addition of the catalyst, a pressure gradient from 5 bar to 1 bar was established, the pressure being reduced by one bar per hour. At a pressure of 5 bar, addition of the isopropyl alcohol was started. 1.5 kg 99.9% isopropyl alcohol were added per hour. An isopropyl alcohol/water distillate was continuously obtained during the reaction. It was passed through the dephlegmator and, after the dephlegmator, was totally condensed and collected. The dephlegmator temperature was reduced with the fall in pressure.

TABLE 8
pressure and temperature during the reaction

| Reactor pressure | Dephlegmator temp. |
|---|---|
| 5 bar | 130° C. |
| 4 bar | 122° C. |
| 3 bar | 114° C. |
| 2 bar | 102° C. |
| 1 bar | 85° C. |

The conversion was determined via the add value. Samples were therefore taken hourly for determination of the acid value. The reaction was continued for ca. 8 h, after which the final acid value should be below 2. The reaction was then terminated. The excess isopropyl alcohol was distilled off (reactor temperature 200° C., dephlegmator temperature 100° C., vacuum gradient from 1000 mbar to 500 mbar in 30 mins.). All the distillate collected was delivered to the enzymatic pre-esterification.

TABLE 9
conversion control

| Reaction | IPA added | Conversion |
|---|---|---|
| time in h | [kg] | [%] |
| 1 | 1.5 | 82 |
| 2 | 3 | 85 |
| 3 | 4.5 | 87 |
| 4 | 6 | 89 |
| 5 | 7.5 | 90 |
| 6 | 9 | 92 |
| 7 | 10.5 | 96 |
| 8 | 12 | 98 |
| 9 | 13.5 | 99.5 |

After termination of the reaction, the excess isopropyl alcohol was distilled off (reactor temperature 200° C., dephlegmator temperature 100° C., vacuum gradient from 1000 mbar to 500 mbar in 30 mins.). All the distillate collected was delivered to the enzymatic pre-esterification. Analysis of the distillate revealed the following composition:

5% water

### 94% IPA

1% fatty acid, IP ester mixture

1-13. (canceled)

14. A process for the production of one or more fatty acid esters, wherein
(a) one or more fatty acids are treated in the presence of one or more esterases with one or more water-containing aliphatic alcohols, each of which alcohols has a boiling point between 60 and 120° C. to produce a pre-esterification product;
(b) water and unreacted alcohol(s) are removed from the pre-esterification product;
(c) the pre-esterification product is subjected to a second, chemically-catalyzed esterification with the same aliphatic alcohol(s) as in step (a) and one or more chemical catalysts; and
(d) the water-containing alcohol removed from step (c) is re-used in step (a).

15. A process according to claim 14, wherein the one or more fatty acids is/are carboxylic acids with the general formula R—COOH, where R is a linear or branched, alkyl or alkenyl group, optionally hydroxysubstituted and with 6 to 32 carbon atoms, which contains up to six conjugated or unconjugated double bonds.

16. A process according to claim 15, wherein the one or more fatty acids is/are selected from the group consisting of caproic acid, oenanthic acid, caprylic acid, pelargonic acid capric acid lauric acid, lauroleic acid, myristic acid, myristoleic acid, palmitic acid, palmitoleic acid, stearic acid, petroselic acid, petrolelaidic acid, oleic acid elaidic acid, ricinoleic acid, linoleic acid linolaidic acid linolenic acid, elaeosstearic acid, arachic acid, gadoleic acid, arachidonic acid, behenic acid, erucic acid, brassidic acid, clupanodonic acid lignoceric acid, serotic acid, melissic acid, eicosapentaenoic acid, docosahexaenoic acid, conjugated linoleic acid, isostearic acids, 2-ethylhexanoic acid, and mixtures of two or more thereof.

17. A process according to claim 14, wherein the one or more fatty acids is/are di- and/or polycarboxylic acids with linear or branched, alkyl or alkenyl chains that are optionally hydroxysubstituted and that contain 2 to 32 carbon atoms.

18. A process according to claim 14, wherein a 1 to 5-fold molar excess of one or more aliphatic alcohols, each with a boiling point of 60 to 120° C., is present during step (a), and a 1 to 4-fold molar excess of one or more such aliphatic alcohols is present during step (d).

19. A process according to claim 18, wherein the one or more aliphatic alcohols are selected from the group consisting of methanol, ethanol, propanol, isopropyl alcohol, 1-butanol, isobutyl alcohol, sec.butyl alcohol, tert.butyl alcohol, and mixtures of two or more thereof.

20. A process according to claim 19, wherein the aliphatic alcohol is isopropyl alcohol.

21. A process according to claim 14, wherein one or more esterases in free or immobilized form are used in step (a).

22. A process according to claim 21, characterized in that the one or more esterases originate from organisms selected from the group consisting of Thermomyces lanugenosus, Candida antarctica A, Candida antarctica B, Rhizomucor miehei, Candida cylindracea, Rhizopus javamicus, Porcine pancreas, Aspergillus niger, Candida rugosa, Mucor javanicus, Pseudomonas fluorescens, Rhizopus oryzae, Pseudomonas sp., Chromobacterium viscosum, Fusarium oxysporum, Penicilium camemberti, and mixtures of two or more thereof.

23. A process according to claim 22, wherein the one or more esterases are one or more lipases.

24. A process according to claim 23, wherein a lipase from Candida antarctica in immobilized form is used as the lipase.

25. A process according to claim 14, wherein one or more inert organic solvents are added to the process.

26. A process according to claim 14, characterized in that the one or more chemical catalysts in step (c) is/are selected from the group consisting of tin(II) compounds, zinc compounds, sulfuric acid, p-toluenesulfonic acid, acidic ion exchangers and mixtures of two or more thereof.
